(19) United States
(12) Patent Application Publication
(10) Pub. No.: US 2016/0228009 A1
(43) Pub. Date: Aug. 11, 2016

(54) OBJECT INFORMATION ACQUIRING APPARATUS

(71) Applicant: CANON KABUSHIKI KAISHA, Tokyo (JP)

(72) Inventors: Toshinobu Tokita, Yokohama-shi (JP); Masato Yajima, Tokyo (JP)

(73) Assignee: CANON KABUSHIKI KAISHA, Tokyo (JP)

(21) Appl. No.: 15/023,034

(22) PCT Filed: Sep. 5, 2014
(86) PCT No.: PCT/JP2014/074150

§ 371 Date: Mar. 18, 2016

(30) Foreign Application Priority Data
Sep. 30, 2013 (JP) 2013-204512

(51) Int. Cl. A61B 5/00

(52) U.S. Cl. CPC A61B 5/0095; A61B 5/742; A61B 5/6843; A61B 2560/0223

(57) ABSTRACT

Provided is an object information acquiring apparatus having: a light source; an optical transmission system that transmits light from the light source; a photoacoustic probe including an irradiating end that irradiates the light onto an object and a receiver that receives an acoustic wave generated from the object, onto which the light is irradiated; a processor that acquires information on the object interior on the basis of the acoustic wave; a display device that displays information on the object interior; a holder that stores the photoacoustic probe and covers the irradiating end; and a light quantity meter that measures quantity of irradiated light, wherein the irradiating end can irradiate light in a state being cover by the holder.

### TECHNICAL FIELD

The present invention relates to an object information acquiring apparatus.

### BACKGROUND ART

Photoacoustic tomography (hereafter “PAT”) is receiving attention as a method specifically for imaging angiogenesis caused by cancer. PAT is a method for irradiating an illumination light (near infrared) onto an object, receiving a photoacoustic wave that is emitted from inside the object using an ultrasonic probe, and generating an image of the received photoacoustic wave.

FIG. 7 is a schematic diagram of a hand held photoacoustic apparatus described in Non-patent Literature 1. A photoacoustic probe 104 is configured such that a receiver 106, for receiving a photoacoustic wave, is fixedly sandwiched by an illumination optical system that includes the irradiating ends 103b of bundle fibers 103.

An illumination light from a light source 101 enters an incident end 103a of the bundle fibers 103. The illumination light propagating through the bundle fibers 103 is irradiated onto the object via the irradiating ends 103b. Then a photoacoustic wave is generated from the object by the photoacoustic effect, and is received by the receiver 106. A processor 107 of an ultrasonic apparatus (US) performs amplification, digitization and image reconstruction on an electric signal converted from the received signal. The constructed image information (IMG) is sent to a monitor 108, which is a display device, and is displayed as a photoacoustic image.

### CITATION LIST

### Non-Patent Literature

NPL 1: S. A. Ermilov et al., “Development of laser optoacoustic and ultrasonic imaging system for breast cancer utilizing handheld array probes”, Photons Plus Ultrasound: Imaging and Sensing 2009, Proc. of SPIE vol. 7177, 2009.

### SUMMARY OF INVENTION

### Technical Problem

The prior art, however, has the following problems.

If the photoacoustic probe 104 disclosed in Non-patent Literature 1 emits illumination light when measurement is not being performed (that is, when the irradiating ends 103b are not contacting the object), the illumination light having a relatively large energy density is emitted into air. Therefore some improvement, such as covering at least the irradiating ends of the photoacoustic probe, is still required when measurement is not being performed.

Further, even if the total quantity of light that is irradiated from the irradiating ends of the photoacoustic probe 104 drops due to deterioration of the light source and problems with the optical transmission system while repeating photoacoustic measurement, in some cases this state cannot be detected.

The drop in the light quantity due to deterioration of the light source 101 can be detected if a light quantity sensor (not illustrated) is disposed between the light source 101 and the incident end 103a.

With this method, however, a drop in the total quantity of light due to problems with the optical transmission system, such as partial disconnection of the bundle fibers 103 and displacement of an optical element (not illustrated), cannot be detected. So the photoacoustic signal may be handled by regarding the total quantity of light as high, even if the total quantity of light is actually low. As a result, the volume of the data and images acquired by correcting the photoacoustic signal with the quantity of light, such as data on an absorption coefficient of an absorber, which is a source of the photoacoustic signal, becomes less than actual data and images, which means that the reliability of the data and the image is diminished.

These problems are not unique to photoacoustic imaging, but are common to optical imaging using relatively large energy density, such DOI (Diffuse Optical Imaging).

With the foregoing in view, it is an object of the present invention that in the photoacoustic probe which irradiates illumination light, the quantity of light that leaks when measurement is not performed is decreased, so that the safety of the testee and the operator and the reliability of data are improved.

### Solution to Problem

The present invention provides an object information acquiring apparatus comprising:
a light source;
an optical transmission system configured to transmit light from the light source;
a photoacoustic probe configured to include an irradiating end for irradiating the light onto an object and a receiver for receiving an acoustic wave generated from the object, onto which the light has been irradiated;
a processor configured to acquire information on the object interior on the basis of the acoustic wave;
a holder configured to store the photoacoustic probe and cover the irradiating end; and
a light quantity meter configured to measure quantity of irradiated light, wherein
the irradiating end can irradiate light in a state of being covered by the holder.

### Advantageous Effects of Invention

According to the present invention, in the photoacoustic probe which irradiates the illumination light, the quantity of light that leaks when measurement is performed can be decreased, so that the safety of the testee and the operator and the reliability of data are improved.

Further features of the present invention will become apparent from the following description of exemplary embodiments with reference to the attached drawings.

### BRIEF DESCRIPTION OF DRAWINGS

FIG. 1 is a diagram depicting a configuration of a photoacoustic apparatus according to an embodiment of the present invention;

FIG. 2A is a diagram depicting a photoacoustic probe according to Example 1 of the present invention;

FIG. 2B is a flow chart depicting an operation according to Example 1 of the present invention;

FIG. 2C is a flow chart depicting an operation according to Example 1 of the present invention;

FIG. 3A is a diagram depicting a photoacoustic probe according to Example 2 of the present invention;

FIG. 3B is a flow chart depicting an operation according to Example 2 of the present invention;

FIG. 4 is a diagram depicting a photoacoustic probe according to Example 3 of the present invention;

FIG. 5A is a diagram depicting a photoacoustic probe according to Example 4 of the present invention;

FIG. 5B is a diagram depicting the photoacoustic probe according to Example 4 of the present invention;

FIG. 6 is a flow chart depicting a method for using a photoacoustic apparatus according to Example 5 of the present invention; and

FIG. 7 is a diagram depicting a configuration of a photoacoustic apparatus according to a prior art.

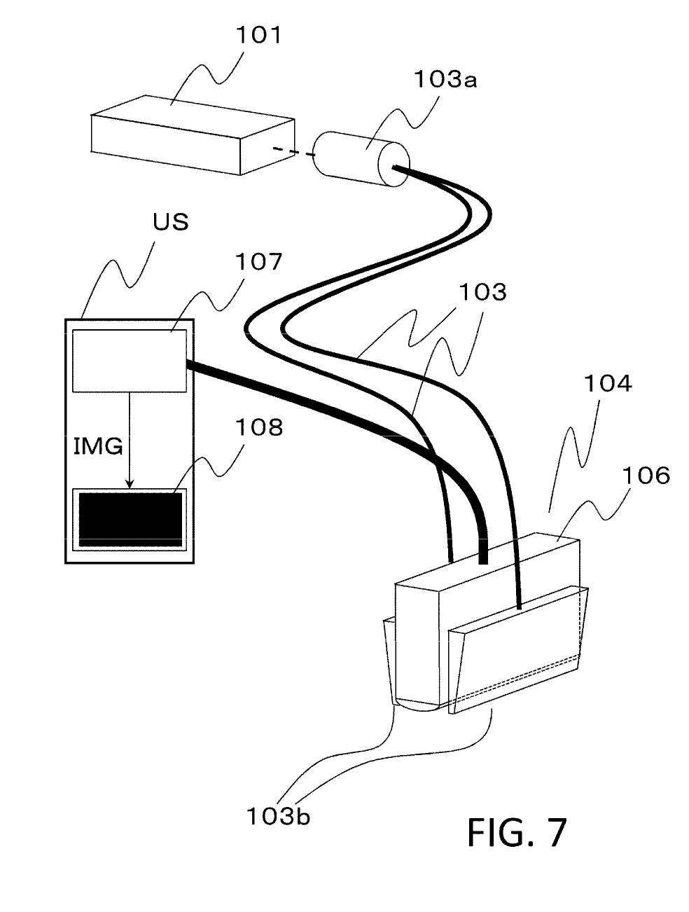

### DESCRIPTION OF EMBODIMENTS

Preferred embodiments of the present invention will now be described with reference to the drawings. Dimensions, materials, shapes of the components described hereinbelow and the relative arrangement thereof should be appropriately changed depending on the configuration and various conditions of the apparatus to which this invention is applied, and are not intended to limit the scope of the invention to the following description.

In the present invention, the acoustic wave includes an elastic wave or a compressional wave called a sound wave, ultrasound wave, photoacoustic wave or light-inducted ultrasound wave. An object information acquiring apparatus of the present invention is a photoacoustic tomography apparatus that irradiates light (electromagnetic wave) onto an object, receives an acoustic wave that is generated inside the object according to a photoacoustic effect, and acquires characteristic information on the object interior.

The characteristic information acquired by the photoacoustic tomography is object information reflecting the initial sound pressure of an acoustic wave generated by the light irradiation, the light energy absorption density and the absorption coefficient derived from the initial sound pressure, and the concentration of a substance constituting the tissue or the like.

The concentration of a substance is, for example, oxygen saturation, oxyhemoglobin concentration or deoxyhemoglobin concentration. The generated characteristic information may be stored and/or used as numeric data, distribution information on each position inside the object, or image data to display an image.

The present invention will now be described with reference to the drawings. Same composing elements are denoted with a same reference symbol, for which redundant description may be omitted. The present invention can also be regarded as an object information acquiring apparatus and an operation method and control method thereof. The present invention can also be regarded as a program for an information processor or the like to execute the control method.

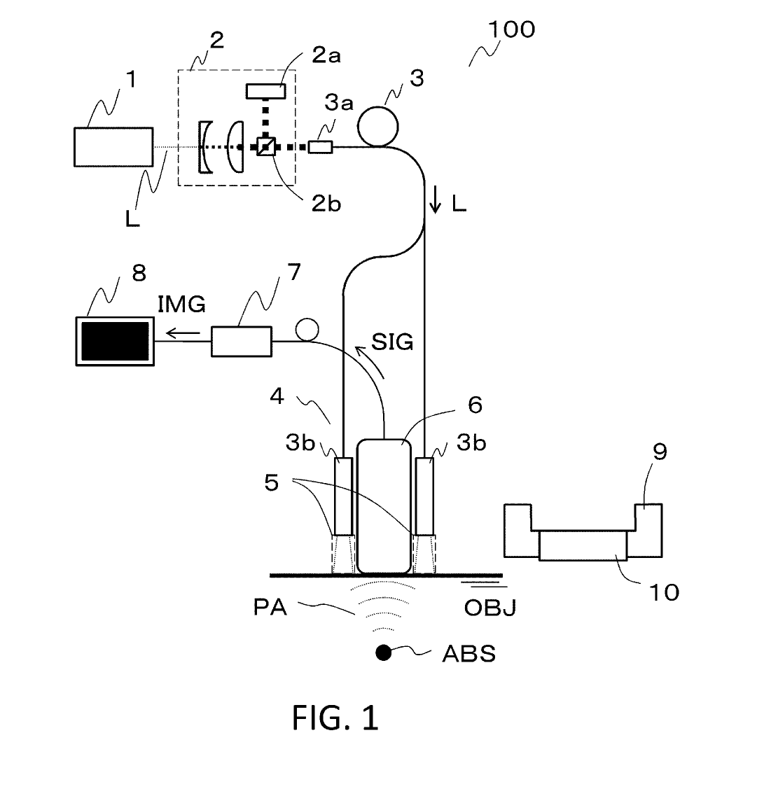

An embodiment of the present invention will be described with reference to FIG. 1 to FIG. 3.

FIG. 1 is a schematic diagram of a photoacoustic apparatus 100. A light source 1 irradiates illumination light (L). A first illumination optical system 2 shapes the illumination light and allows it to enter an incident end 3a of a bundle fiber 3. The bundle fiber 3 transmits the illumination light to a photoacoustic probe 4, and irradiates the illumination light from the irradiating ends 3b thereof.

The photoacoustic probe 4 includes irradiating ends 3b of the bundle fiber 3, a second illumination optical system 5 that shapes illumination light irradiated from the irradiating ends 3b, and a receiver 6 that receives the photoacoustic wave. The light irradiated through the second illumination optical system 5 onto an object (OBJ) is diffused inside the object, and an absorber (ABS) that absorbed this light emits a photoacoustic wave (RA).

The photoacoustic wave is converted into an electric signal (SIG) by such an element as a piezoelectric element or CMUT enclosed in the receiver 6, and this electric signal is sent to a processor 7. The processor 7 amplifies the electric signal and generates image information (IMG) through a digital convertor or a filter, and displays the image on a display device 8. For the processor 7, an information processor that includes a CPU, a memory, and a processing circuit, which performs various processings for example, can be used.

In FIG. 1, the bundle fiber 3 branches along its path, and the irradiating end 3b and the second illumination optical system 5 are disposed at two locations. However, a number of branches is not limited to two. The irradiating end 3b may be disposed adjoining only one side of the receiver 6 without branching the bundle fiber 3.

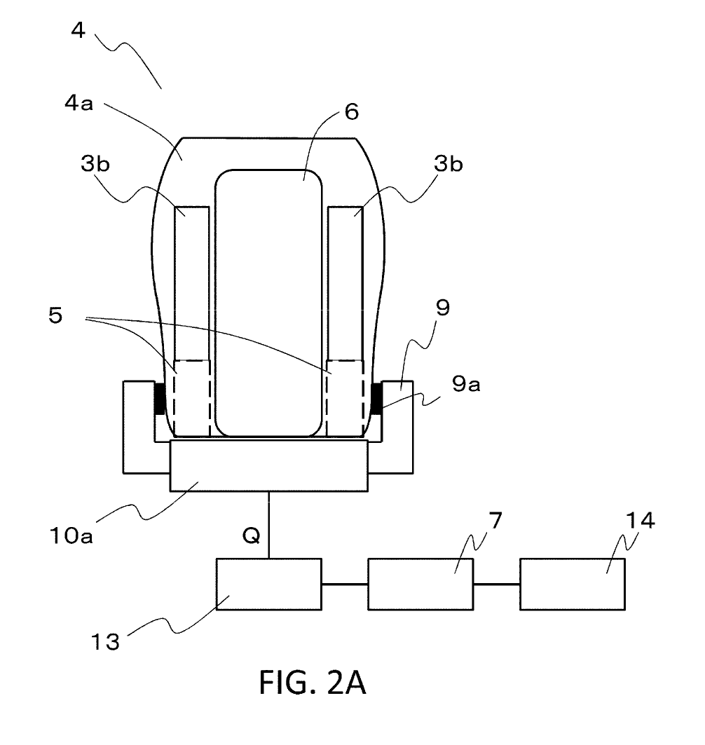

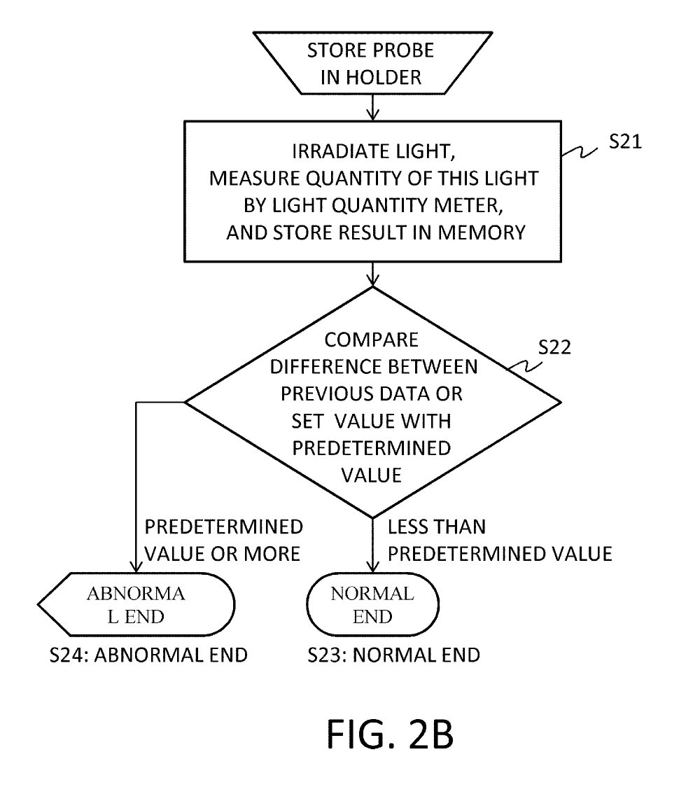

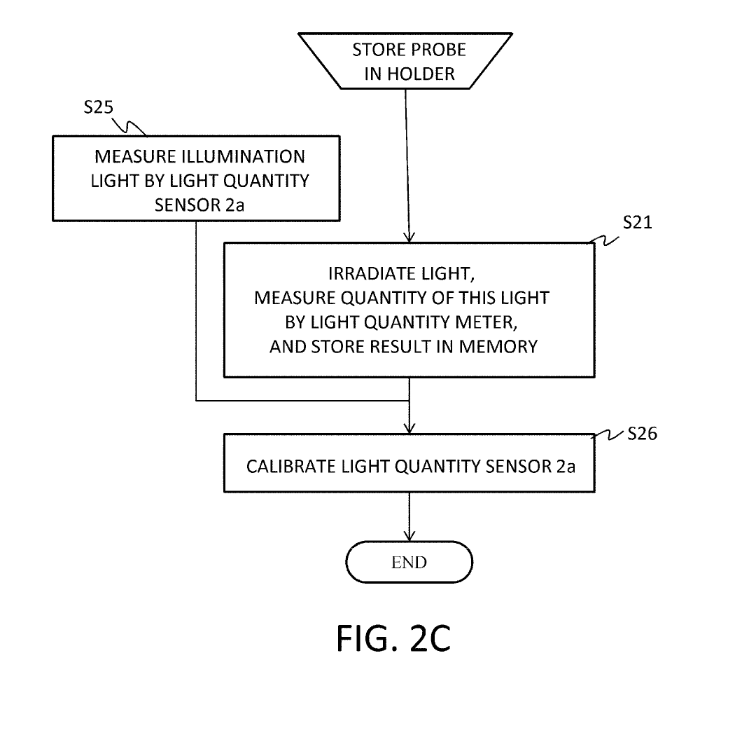

It is preferable that the photoacoustic probe 4 is covered by a housing 4a as illustrated in FIG. 2A.

For the light source 1, a light source that emits near-infrared having a 600 nm to 1100 nm wavelength is preferable. For example, a pulsed laser, such as an Nd:YAG laser or an alexandrite laser, is used. A Ti: sa laser or an OPO laser, of which excitation light is an Nd:YAG laser light, may be used.

In FIG. 1, the light from the light source 1 is transmitted via the first illumination optical system 2 and the bundle fiber 3. However, the optical transmission system is not limited to this. For example, the optical transmission system may be an optical transmission system where a mirror, a prism or the like are combined, and reflection and refraction thereof are used. Further, as the optical transmission system, the light source 1 may be a semiconductor laser disposed at the irradiating ends 3b.

Irradiation of the illumination light and the reception of the photoacoustic wave by the receiver 6 must be synchronized. For this synchronization, a possible method is that either one of the paths from the light source 1 to the second illumination optical system 5 may be partially branched, and the signal is detected by a sensor (not illustrated), such as a photodiode. Using this detected signal as a trigger, the receiver 6 can start reception. Another possible method is that the emission timing of the light source 1 and the reception timing of the processor 7 are synchronized using a pulse generator (not illustrated).

In the first illumination optical system 2, not only the optical element that shapes the illumination light, but also a reflection element 2b that reflects several % of illumination light and the light quantity sensor 2a that measures the reflected light thereof, are disposed. For the light quantity sensor 2a, a photodiode, a photomultiplier or the like can be used. In FIG. 1, these blocks are disposed in the first illumination optical system 2. However, the disposing location is not limited to this, but can be inside the light source 1, or anywhere on the path from the light source to the irradiation ends of the photoacoustic probe 4. For the reflection element 2b, a plane parallel glass or a mirror can be used.

Normally a value determined by multiplying the output value of the light quantity sensor 2a by a predetermined scale factor is regarded as a total quantity of light from the irradiating ends of the photoacoustic probe 4. The predetermined scale factor is determined by the reflection efficiency of the reflection element 2b and the light transmission efficiency from the reflection element 2b to the second illumination optical system 5. For example, if the reflection efficiency of the reflection element 2b is 5%, then 95% of the illumination light is transmitted and reaches the incident end 3a of the bundle fiber. The light transmission efficiency is determined by the incident efficiency to the bundle fiber 3, the irradiating efficiency, and the transmittance of the optical element, such as a diffusion plate disposed in the second illumination optical system 5, and here is assumed to be 60%.

Under these conditions, 57% (=0.95×0.6) of the illumination light emitted from the light source 1 is irradiated from the irradiating ends of the photoacoustic probe 4, and this becomes the total quantity of light. In other words, the quantity of light that can be measured by the light quantity sensor 2a, with respect to the total quantity of light, becomes approximately 8.8% (=0.05/0.57). This means that the value determined by multiplying the quantity of light, measured using the light quantity sensor 2a by 11.4 ((=1/0088) can be regarded as the total quantity of light. In this way, a value determined by multiplying the measurement value of the light quantity sensor 2a by a predetermined scale factor is regarded as the total quantity of light used when an object is measured. This scale factor or conversion formula has been stored in the processor 7.

For the conversion formula, a formula by adding an offset component (total quantity of light=11.4×output of light quantity sensor 2a+offset) may be used instead of a formula of simply multiplying a scale factor (total quantity of light=11.4×output of light quantity sensor 2a). Once the total quantity of light that is irradiated onto the surface of the object is known by this formula, the light quantity distribution inside the object can be calculated by setting the boundary conditions using this total quantity of light along with the known irradiation range. To calculate the light quantity distribution inside the object, a light diffusion equation (transport equation) or the Monte Carlo method can be used. Here known values or estimated values are used for background optical constants (μ_{eff}: equivalent attenuation coefficient absorption coefficient; μa: absorption coefficient; and μₛ′: equivalent scattering coefficient) inside the object.

In the present invention, a holder 9 is included for storing the photoacoustic probe 4 when measurement is not performed (when an object is not imaged or measured). A light quantity meter 10 is disposed inside the holder 9 so that the total quantity of light irradiated from the photoacoustic probe 4 can be measured.

Specifically the light quantity meter 10 will be described with reference to FIG. 2A. In the holder 9, a power meter 10a (light quantity meter 10) is disposed to measure the total quantity of light irradiated from the irradiating ends of the photoacoustic probe 4. For the power meter 10a, a photoelectric conversion type, such as a photodiode, or a heat exchange type, such as a thermopile, can be used. The power meter 10a is disposed in a position facing the irradiating ends of the photoacoustic probe 4 in a state where the photoacoustic probe 4 is stored in the holder 9.

By this configuration, the total quantity of light irradiated from the photoacoustic probe 4, that is, the total quantity of light irradiated onto the object, can be measured. The measured value (Q) of the quantity of light is sent to a memory 13.

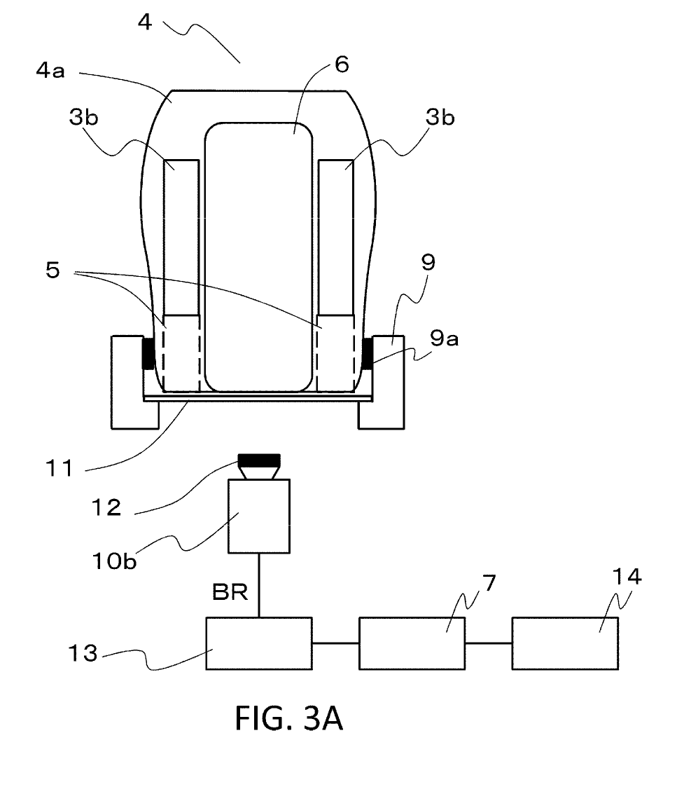

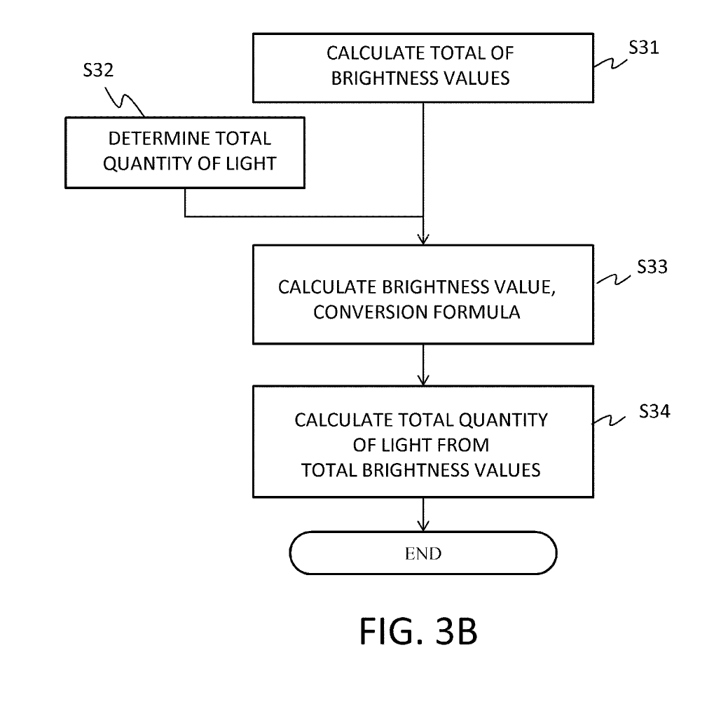

A diffusion plate 11 may be disposed in the photoacoustic probe 4 at a location facing the irradiating ends, as illustrated in FIG. 3A, in a state where the photoacoustic probe 4 is stored in the holder 9. Then the distribution of the quantity of light irradiated onto the diffusion plate 11 is imaged using an infrared camera 10b (light quantity meter 10) via an ND filter 12. If this configuration is used, the total quantity of light irradiated from the photoacoustic probe 4 can not only be measured by totaling the brightness value (BR) of each pixel, which is measured by the infrared camera 10b, but also the in-plane distribution of the quantity of light irradiated from the irradiating ends of the photoacoustic probe 4 can be measured.

In FIG. 2A and FIG. 3A, an elastic body 9a is disposed inside the holder 9. For the elastic body 9a, an easily deformable resin, such as various rubbers and urethane, is suitable. The main body of the holder 9 is constituted by a relatively rigid material, such as a metal, resin (e.g. plastic) or ceramic. If the elastic body 9a is disposed, the gap between the photoacoustic probe 4 and the holder 9 can be filled when the photoacoustic probe 4 is stored in the holder 9. Then the light that leaks from the holder 9 can be reduced when the total quantity of light irradiated from the irradiating ends of the photoacoustic probe 4 is measured by the light quantity meter 10. As a result, safety of the testee and the operator improves.

This embodiment was described using the case of photoacoustic tomography. However application of the present invention is not limited to this. For example, the present invention can also be applied to optical imaging using light of which energy density is relatively high, such as DOI (Diffuse Optical Imaging). This is the same for the following examples.

### Example 1

Now a concrete measurement procedure using the configuration in FIG. 2A will be described with reference to the flow charts in FIG. 2B and FIG. 2C. The output of the power meter 10a is stored in the memory 13. At this time, the processor 7 determines whether the total quantity of light irradiated from the photoacoustic probe 4 is normal or not according to each step of the flow in FIG. 2B.

S21: Light is irradiated from the irradiating ends in a state where the photoacoustic probe 4 is stored in the holder 9, and the quantity of light at this time is measured by the power meter 10a. This measurement result is stored in the memory 13.

S22: The difference between this measurement result and the previous measurement result or the reference total quantity of light (set value) is compared with a predetermined value.

S23: If the difference in S22 is less than the predetermined value, the flow ends normally.

S24: If the difference in S22 is the predetermined value or more, the flow does not end normally (ends abnormally). In this example, the standard total quantity light is 50 mJ, and the threshold of the difference is ±5 mJ.

According to this procedure, safety is maintained by storing the photoacoustic probe in the holder, so that the actual quantity of irradiated light can be measured in a safe state. As a result, problems with the apparatus can be quickly detected.

It is preferable that the apparatus includes a presentation unit 14 to present the determination result in S22. For the presentation unit 14, an LED that presents the state by lighting or blinking, or a unit that notifies the state by voice can be used. The display device 8 may be used as the presentation unit 14, where the state is presented by characters or images. This allows the operator to quickly recognize the changes of the total quantity of light irradiated from the photoacoustic probe 4, therefore measurement under poor conditions can be prevented.

Here the power meter 10 can measure optical energy that is actually irradiated onto the object. The light quantity sensor 2a enclosed in the first illumination optical system 2, on the other hand, measures several percent of the light irradiated from the light source 1, and cannot measure the optical energy irradiated onto the object. However as described above, the value measured by the power meter 10 and the value measured by the light quantity sensor 2a have a proportional relationship. Therefore the light quantity sensor 2a can be calibrated based on these measurement results. FIG. 2C shows the flow of calibration.

S21: Light is irradiated from the irradiating ends in a state where the photoacoustic probe 4 is stored in the holder 9, and the quantity of light at this time is measured by the power meter 10a. This measurement result is stored in the memory 13 (the same as S21 in FIG. 2B).

S25: Along with S21, the illumination light is measured by the light quantity sensor 2a.

S26: A calibration value to determine the total quantity of light using the light quantity sensor 2a (conversion formula) is calculated based on the output values of the power meter 10 and the light quantity sensor 2a.

If the light quantity sensor 2a is calibrated and the total quantity of light irradiated from the photoacoustic probe 4 can be converted as above, then the total quantity of light in an actual photoacoustic measurement can be known.

### Example 2

In this example, an infrared camera 10b illustrated in FIG. 3A is used as the light quantity meter 10. It is difficult to directly image the illumination light irradiated from the irradiating ends of the photoacoustic probe 4 using the infrared camera 10b. Hence a diffusion plate 11 is disposed in a position facing the irradiating ends of the photoacoustic probe 4, so as to adjust the focal point of the infrared camera 10b to the diffusion plate 11.

If the quantity of illumination light irradiated from the photoacoustic probe 4 is high, the brightness value of each pixel imaged by the infrared camera 10b may become saturated, or an image receiving element of the infrared camera 10b may be damaged. To prevent this, it is preferable that an ND filter 12 is disposed between the diffusion plate 11 and the infrared camera 10b.

A method for determining the total quantity of light by totaling the brightness value of each pixel imaged by the infrared camera 10b will be described with reference to the flow chart in FIG. 3B.

S31: The total of the brightness values measured by the infrared camera 10b is calculated. For example, the brightness value is represented by 256 gradations, and the brightness values of 1280×960 pixel data are totaled.

S32: The total quantity of light from the irradiating ends of the photoacoustic probe 4 is determined. The total quantity of light is determined according to the method described in Example 1. In other words, a method for determining a conversion value into the total quantity of light using the calibrated light quantity sensor 2a (FIG. 1) or a method for directly measuring the total quantity of light by the power meter 10a is used.

S33: The brightness value is calibrated and a conversion formula is created based on the total of the brightness values and the total quantity of light.

S34: The total quantity of light is determined based on the total of the brightness values.

In S34, the light quantity distribution in the irradiation plane of the light irradiated from the irradiating ends of the photoacoustic probe 4 can also be determined from each pixel value of the infrared camera 10b. Thereby the light quantity distribution on the surface of the object is known, hence if the light quantity distribution is used as a boundary condition when the light quantity distribution inside the object is calculated, the calculation accuracy thereof can be further increased.

As described above, the total quantity of light from the photoacoustic probe stored in the probe can be safely measured by the method of this example as well.

### Example 3

The method for measuring the light quantity distribution in the irradiation plane, of the light irradiated from the irradiating ends of the photoacoustic probe 4 is not limited to the method of using the infrared camera 10b described in Example 2. In this example, a configuration and a method when the light quantity distribution is measured by the power meter 10a will be described.

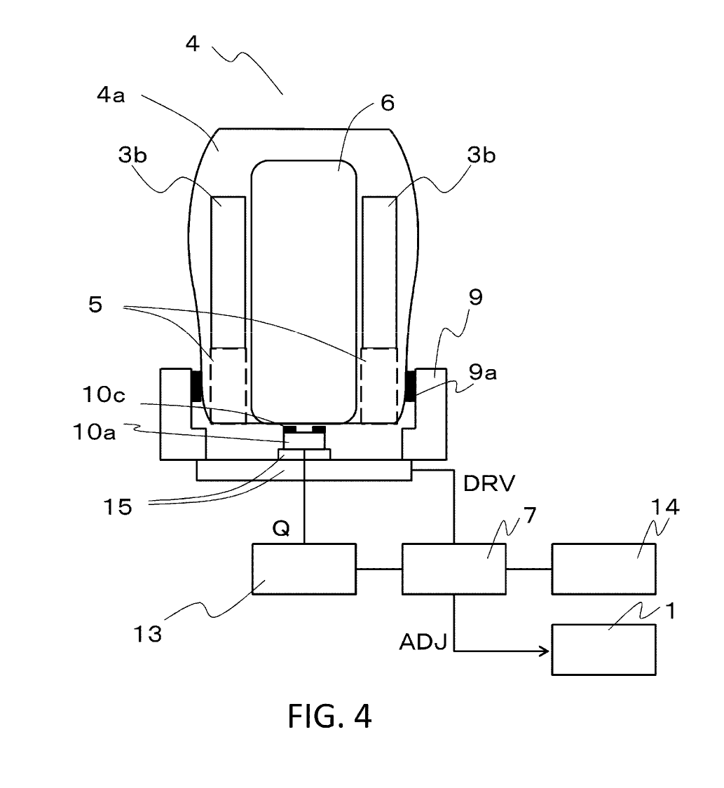

The power meter 10a of Example 1 has an area large enough to face the entire irradiating ends of the photoacoustic probe 4. In Example 3, on the other hand, a power meter 10a having a small area is scanned, as illustrated in FIG. 4A. Therefore the power meter 10a having a small area is mounted on an XY stage 15. Then the light quantity distribution in the irradiation plane of the light irradiated from the irradiating ends of the photoacoustic probe 4 can be measured using a relatively inexpensive power meter 10a having a small area, without using the infrared camera 10b.

ANSI Z136.1-2000 specifies that a 3.5 mm diameter area should be measured to determined whether irradiated energy per unit area exceeds the MPE (Maximum Permissible Exposure). Therefore if the measurement area of the power meter 10a is set to a 3.5 mm diameter area, or if an aperture 10c having a 3.5 mm diameter opening is formed on the power meter 10a, then the irradiated energy per unit area can be measured by a method conforming to ANSI Z136.1-2000.

To ensure safety to human skin, the processor 7 determines whether the energy density measured by the power meter 10a exceeds a predetermined value. For the predetermined value, a value that is about 0.8 times the MPE for human skin is used considering safety factors. If the processor 7 determines that the irradiated energy density exceeded the predetermined value, the processor 7 makes an adjustment to lower the illumination intensity of the light source 1 (adjustment instruction ADJ). Thereby the optical energy density is kept at or below the predetermined value, and safety can be ensured.

Other methods that can be used to keep the optical energy density at MPE or less are, for example, inserting a filter between the light source 1 and the irradiating ends of the photoacoustic probe 4, or inserting diffusion plates having a wide diffusion angle in the second illumination optical system 5.

The XY stage 15 in FIG. 4 is disposed to move the power meter 10a for scanning. The XY stage 15 is operated by a drive instruction (DRV) from the processor 7. The scanning mechanism however is not limited to this. Conversely for example, the photoacoustic probe 4 stored in the holder 9 may be moved for scanning. In other words, it is required to perform a relative scan between the irradiating end of the photoacoustic probe 4 and the measurement surface of the power meter 10a in the in-plane direction.

In Example 3, by moving the power meter 10a for scanning, the light quantity distribution in the plane where the light is irradiated from the irradiating ends of the photoacoustic probe 4 is measured. Thereby not only can the total quantity of light be measured safely, but also the light quantity distribution inside the object can be calculated. Further, use of a relatively inexpensive power meter can reduce cost.

Furthermore, the processor 7 in Example 3 determines whether the energy density exceeds a predetermined value, and adjusts the energy density if it is exceeded. Thereby the safety of the operator and the testee can be ensured. For this purpose, the infrared camera in Example 2 may be used as the light quantity meter 10, instead of the compact power meter 10a.

### Example 4

An illumination light having several tens mJ to a hundred and several tens mJ of high energy is irradiated from the irradiating end of the photoacoustic probe 4 through a relatively small area. Therefore even if the MPE for human skin described in Example 3 is not exceeded, the MPE for a human retina, of which reference value is lower, may be exceeded. Therefore it is desirable that a mechanism, which does not allow irradiation of light when the photoacoustic probe 4 is not in contact with the object, is disposed near the irradiating ends of the photoacoustic probe 4 to ensure the safety of the testee and the operator.

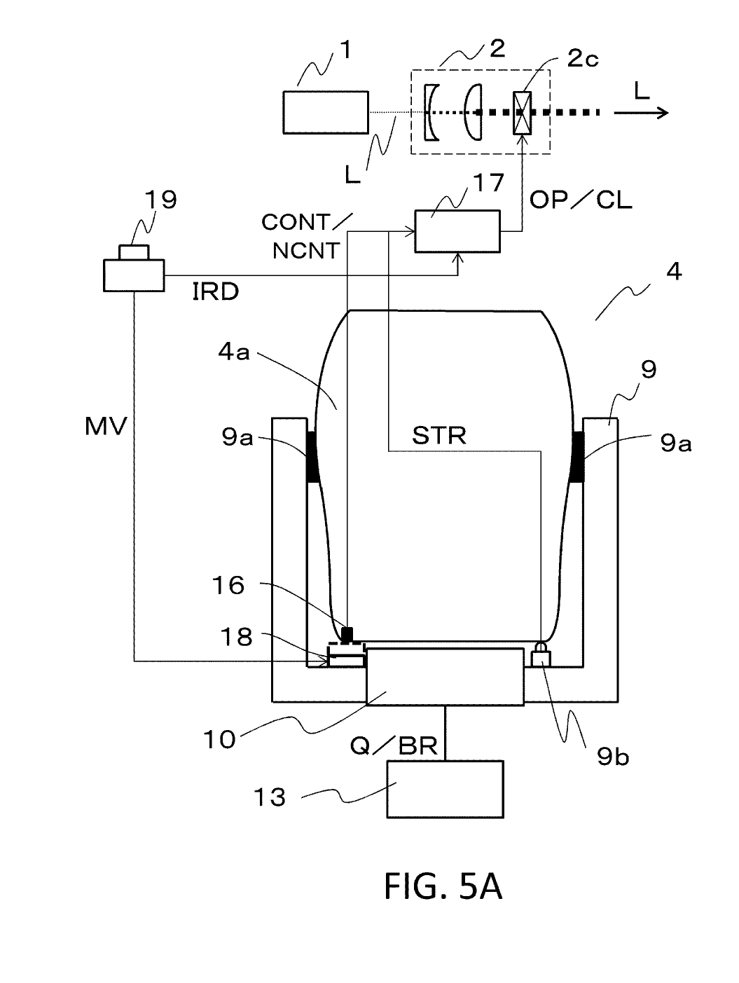

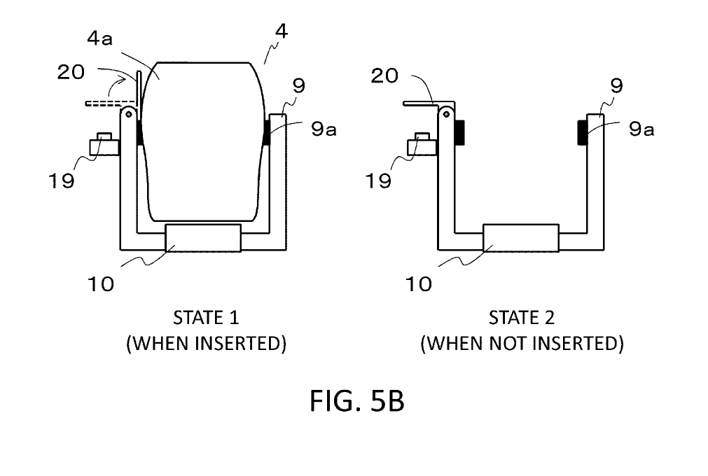

In this example, a contact detection sensor 16 is disposed outside the irradiating end of the photoacoustic probe 4, as illustrated in FIG. 5A. For the contact detection sensor 16, an optical, an electrostatic or a mechanism sensor, or a strain gauge can be used. A contact may be detected by transmitting and receiving the ultrasound wave by the receiver 6 (FIG. 1). The contact detection sensor 16 outputs contact information (CONT) if the irradiating ends are in contact with the object, or non-contact information (NCNT) if the irradiating ends are not in contact with the object.

A controller 17 outputs a shutter open/close instruction (OP/CL) in accordance with the contact/non-contact information. In other words, if the non-contact information is outputted, the controller 17 closes a shutter 2c in the first illumination optical system 2, or closes an internal shutter (not illustrated) inside the light source 1. If the light source 1 is a Q switch laser, the controller 17 stops the Q switch. By this method, the controller 17 controls so that the illumination light (L) is not irradiated from the irradiating ends of the photoacoustic probe 4.

If the contact information is outputted, on the other hand, the controller 17 controls so that the illumination light can be irradiated from the irradiating ends of the photoacoustic probe 4. In other words, the controller 17 opens the shutter 2c or the internal shutter inside the light source 1, or turns the Q switch ON if the light source 1 is a Q switch laser.

By this configuration, safety when the object and the probe are not contacted can be ensured. However when the total quantity of light is measured in a state where the photoacoustic probe 4 is stored in the holder 9 according to this invention, a problem could occur depending on the shape of the holder 9.

In other words, if there is a space (gap) between the contact detection sensor 16 and a portion inside the holder 9 that faces the contact detection sensor 16, the contact detection sensor 16 outputs the non-contact information. In this case, even if the operator presses the irradiation switch 19, the illumination light is not irradiated from the irradiating ends of the photoacoustic probe 4 due to the control of the controller 17. Hence the light quantity meter 10 cannot execute measurement. This means that in a state where the photoacoustic probe 4 is normally stored in the holder 9, the contact detection sensor 16 must output the contact information. The configuration and the method for this will now be described.

In the first example, even if there is a space between the contact detection sensor 16 and a portion inside the holder 9 that faces the contact detection sensor 16, the controller 17 forcibly allows irradiating the light if the irradiation switch 19 is pressed. In other words, if the irradiation switch 19 is pressed, the irradiation instruction (IRD) is outputted to the controller 17.

In this case however, the illumination light is irradiated if the irradiation switch 19 is pressed, even if the photoacoustic probe 4 is not stored in the holder 9 normally. Therefore it is preferable to prompt the operator to store the photoacoustic probe 4 in the holder 9 by disposing the irradiation switch 19 next to the holder 9.

It is even more preferable to dispose a cover 20, as illustrated in FIG. 5B, so that the irradiation switch 19 can be operated only when the photoacoustic probe 4 is stored in the holder 9. Because a cover 20 exists, the switch cannot be operated unless the photoacoustic prove 4 is stored in the holder 9. When the photoacoustic probe 4 is stored in the holder 9, the illumination light cannot be irradiated unless the irradiation switch 19 is pressed.

In the next example, the gap between the contact detection sensor 16 and the portion inside the holder 9, that faces the contact detection sensor 16, is narrowed until the contact detection sensor 16 can detect the state as “contact”. If this method is used, the shape inside the holder, the shape of the housing 4a of the probe, and the shape of the elastic body 9a are adjusted respectively so that the gap between the photoacoustic probe 4 and the holder 9 is filled by the elastic body 9a.

Then if the storing state of the photoacoustic probe 4 is insufficient, the contact information is not outputted, and the illumination light is not irradiated. If the photoacoustic prove 4 is correctly stored in the holder 9, the quantity of light that leaks from the holder 9 is controlled even if the illumination light is irradiated, since the gap between the photoacoustic probe 4 and the holder 9 is filled by the elastic body 9a.

In another example, as illustrated in FIG. 5A, a moving member 18 is disposed inside the holder 9, facing the contact detection sensor 16. When the irradiation switch 19 is pressed, the moving member 18 can be moved to a position where the contact detection sensor 16 can detect the state as “contact” (executing a moving member operation instruction MV). Then when the photoacoustic probe 4 is stored in the holder 9, the illumination light is not irradiated unless the irradiation switch 19 is pressed.

By the configurations and methods described above, the illumination light can be irradiated in a state where the photoacoustic probe 4 is correctly stored in the holder 9. Then as described in Example 1 to Example 3, the total quantity of light irradiated from the irradiating ends of the photoacoustic probe 4 can be measured using the light quantity meter 10.

A storing detection sensor 9b, which outputs storing information (STR) if it is detected that the photoacoustic probe 4 is stored in the holder 9, may be disposed inside the holder 9. If the storing information is received, the controller 17 enables irradiation of the illumination light (e.g. opening of shutter 2c). For the storing detection sensor 9b, not only a mechanical type but also an optical or electrostatic type switch can be used.

Then the light quantity meter 10 can measure the total quantity of light only when the photoacoustic probe 4 is stored in a predetermined position inside the holder 9. If the storing detection sensor 9b is disposed in a plurality of locations, the total quantity of light can be measured only when the irradiating end of the photoacoustic probe 4 and the light quantity meter are parallel, whereby the measurement conditions of the light quantity meter 10 can be reproduced, and measurement accuracy improves.

The configurations and control methods described above may be used by itself or in combination. Thereby the illumination light is not irradiated when the irradiating ends of the photoacoustic probe 4 are not in contact with the object, hence the quantity of light that leaked can be decreased, and safety of the testee and the operator can be ensured. If the photoacoustic probe 4 is stored in a predetermined position in the holder 9, the light is irradiated and the quantity of light can be measured.

### Example 5

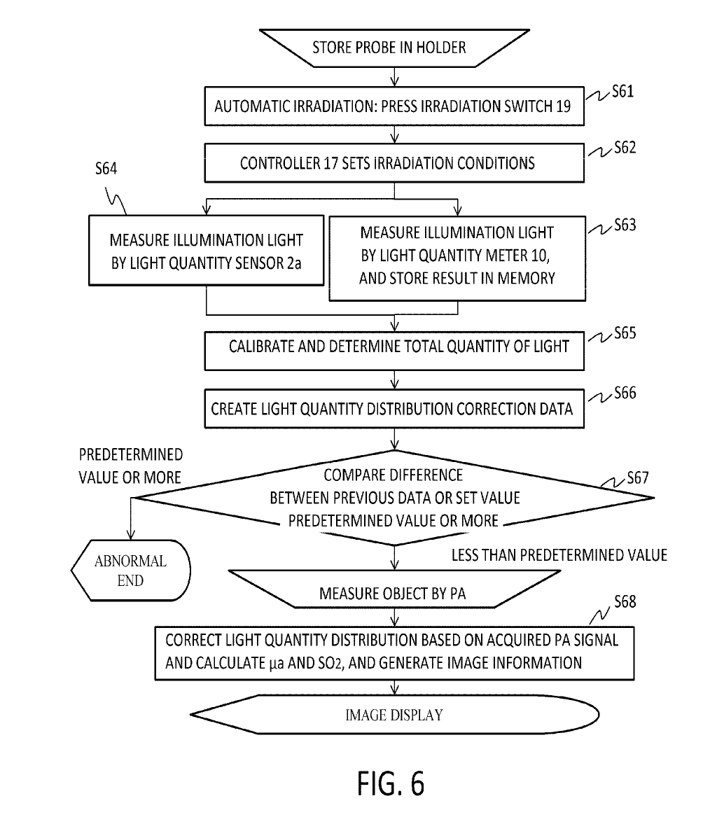

A method for using the photoacoustic apparatus 100 including the photoacoustic probe 4 and the holder 9 described in Example 4 will be described with reference to the flow chart in FIG. 6. The apparatus according to this example includes the infrared camera 10b (light quantity meter 10), the diffusion plate 11 and the ND filter 12 in FIG. 3A, and various switches and sensors in FIG. 5A.

When the apparatus is started up, or when measurement is not performed, the photoacoustic probe 4 is stored in the holder 9.

S61: When the apparatus is started up, the controller 17 performs the automatic irradiation sequence. When the operator stores the photoacoustic probe 4 in the holder 9, the operator presses the irradiation switch 19, whereby the controller 17 performs the sequence for irradiation (S62).

S62: If the contact detection sensor 16 is disposed in the photoacoustic probe 4, the conditions under which the contact detection sensor 16 can detect the state as “contact” are set. For example, the moving member 18 is moved is illustrated in FIG. 5. Then the controller 17 sets the irradiation conditions. If the storing detection sensor 9b is in the holder 9, the controller 17 sets the irradiation conditions when the storing detection sensor 9a detects the photoacoustic probe 4. Even if the contact detection sensor 16 and the storing detection sensor 9a do not exist, the controller 17 sets the irradiation conditions after S61.

The irradiation conditions set by the controller 17 are: opening the internal shutter inside the light source 1 and the shutter 2c inside the first optical system 2; and turning the Q switch ON if the light source 1 is a Q switch laser. Thereby the illumination light is irradiated from the irradiating ends of the photoacoustic probe 4. The irradiation time and a number of times of irradiation are programmed in the controller 17, and in this example, the illumination light is irradiated 100 times (10 seconds×10 Hz).

S63: The illumination light irradiated from the irradiating ends of the photoacoustic probe 4 is diffused by the diffusion plate 11, and is imaged by the infrared camera 10b.

S64: Along with S63, the illumination light is measured by the light quantity sensor 2a (FIG. 1) disposed in the second optical system 2. It is assumed that the light quantity sensor 2a has been calibrated for converting the total quantity light. For the calibration method, the method described in Example 1 can be used.

S65: The processor 7 calculates the total brightness value of each pixel imaged in S63, and calibrates the brightness value using the measurement values by the light quantity sensor 2a in S64. Thereby the brightness when the infrared camera 10b images can be calibrated, and the quantity of light can be calculated from the brightness value. Then the total quantity of light from the irradiating ends of the photoacoustic probe 4 is determined using the brightness value of the light quantity sensor 2a or the infrared camera 10b.

S66: The processor 7 determines the boundary condition based on the total quantity of light determined in S65 and the irradiated area on the object, or the light quantity distribution of light irradiated on the surface of the object which is known by the calibrated brightness value. Using this boundary condition, the light quantity distribution of light that enters into the object while being absorbed and scattered is calculated, and light quantity distribution correction data is created.

S67: The total quantity of light and brightness data or the light quantity distribution data inside the object is compared with the corresponding data up to the previous measurement or with the reference set value. In this example, the set value of the total quantity of light is 50 mJ and the predetermined value is 50±5 mJ. If this difference is at or over the predetermined value, that is, if the total quantity of light is 45 mJ or less and 55 mJ or more, this sequence does not end normally (ends abnormally), and an abnormal end state is presented in the presentation unit 14 or a message is displayed on the display device 8.

If the quantity of light drops, it is possible that the quantity of light changed due to the contamination of the irradiating ends of the photoacoustic probe 4 or the diffusion plate 11 (light quantity meter 10), so a “clean and re-measure” message is presented. In the case of re-measurement, a message to start the sequence again from S61 is presented. Further, it is also possible that a problem occurred to the light transmission in the light source 1, the bundle fiber 3 or the like, hence an “abnormal end” message is presented if no improvement occurs after re-measurement. If the total quantity of light is greater than the predetermined range, an appropriate countermeasure is taken.

If the difference is less than the predetermined value, on the other hand, the operator holds the photoacoustic probe 4 and performs photoacoustic measurement on the object. In this way, fluctuation of the total quantity of light or fluctuation of brightness data and light quantity distribution data inside the object, due to the fluctuation of the total quantity of light, can be minimized, therefore a stable photoacoustic measurement result can be acquired.

S68: A photoacoustic image is created from the acquired photoacoustic signal, and this image is displayed on the display device 8. The total quantity of light when the photoacoustic signal was acquired is converted from the measured value of the light quantity sensor 2a. Then the converted total quantity of light and the light quantity distribution correction data in S66, and the light quantity distribution inside the object when the photoacoustic signal was acquired is corrected.

The photoacoustic signal is given by the following Expression (1).

p=Γμaφ (1)

Here p denotes an initial sound pressure of the photoacoustic signal, Γ denotes a Gruneisen coefficient, μa denotes an absorption coefficient, and φ denotes a quantity of light, and the absorption coefficient μa can be determined from the photoacoustic signal (p), the corrected light quantity distribution inside the object (φ) and the Gruneisen coefficient Γ which is about 0.5.

Furthermore, if the wavelength of the light emitted by the light source 1 is variable, the spectral characteristic of the absorber, which is the sound source of the photoacoustic signal, is known. For example, if the absorber is blood (hemoglobin), the oxygen saturation of the hemoglobin can be measured as well. Since the light quantity distribution on the surface of the object, which is the boundary condition used for accurately determining the light quantity distribution inside the object, can be accurately measured, performance in measuring the absorption coefficient μa, the oxygen saturation or the like can be further improved.

The above flow can also be applied to a case of using the power meter 10a as the light quantity meter 10. If the power meter 10a itself has been calibrated, the calibration of the light quantity meter 10 described in S605 is unnecessary, and the light quantity sensor 2a can be calibrated by the method described in Example 1, which is the same as S64.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-204512, filed on Sep. 30, 2013, which is hereby incorporated by reference herein in its entirety.

1. An object information acquiring apparatus comprising:
a light source;
an optical transmission system configured to transmit light from the light source;
a photoacoustic probe configured to include an irradiating end for irradiating the light onto an object and a receiver for receiving an acoustic wave generated from the object, onto which the light has been irradiated;
a processor configured to acquire information on the object interior on the basis of the acoustic wave;
a holder configured to store the photoacoustic probe and cover the irradiating end; and
a light quantity meter configured to measure quantity of irradiated light,
wherein the irradiating end can irradiate light in a state of being covered by the holder.

2. The object information acquiring apparatus according to claim 1,
further comprising a light quantity sensor configured to branch a part of the light and measure the light quantity thereof in an area between the light source and the irradiating end of the photoacoustic probe,
wherein the processor compares a total quantity of light irradiated from the irradiating end that the light quantity meter has measured and the measured value of the light quantity sensor, and performs calibration to convert the measured value of the light quantity sensor into the total quantity of light.

3. The object information acquiring apparatus according to claim 1, wherein
the light quantity meter is a power meter.

4. The object information acquiring apparatus according to claim 1, further comprising a diffusion plate that is disposed in the holder at a position facing the irradiating end, wherein
the light quantity meter is an infrared camera that images light diffused by the diffusion plate, and
the processor determines at least one of the total quantity of light irradiated from the irradiating end and the light quantity distribution in a plane where the light is irradiated, on the basis of a brightness value of each pixel acquired by the imaging by the infrared camera.

5. The object information acquiring apparatus according to claim 4,
wherein the processor performs calibration to convert the brightness value of each pixel of the infrared camera into the total quantity of light irradiated from the irradiating end.

6. The object information acquiring apparatus according to claim 1, further comprising:
a memory configured to store light quantity measured by the light quantity meter and a predetermined value as a reference of the light quantity; and
a presentation unit configured to present information,
wherein the processor compares the measured light quantity and the predetermined value, and presents whether the measured light quantity is normal or not in use of the presentation unit, based on a difference between the measured light quantity and the predetermined value.

7. The object information acquiring apparatus according to claim 1, wherein the holder includes an elastic body that fills a gap when the photoacoustic probe is stored in the holder.

8. The object information acquiring apparatus according to claim 1, further comprising a stage configured to perform a relative scan between the light quantity meter and the irradiating end.

9. The object information acquiring apparatus according to claim 1, wherein the processor decreases the energy density of the light irradiated from the irradiating end when the energy density of the light irradiated from the irradiating end exceeds a predetermined value.

10. The object information acquiring apparatus according to claim 1, further comprising:
an irradiation switch configured to enable an operator to instruct irradiation of the light; and
a controller configured to set conditions of irradiating the light from the irradiating end when the irradiation switch is pressed.

11. The object information acquiring apparatus according to claim 10, further comprising a cover configured to disable operation of the irradiation switch in a state where the photoacoustic probe is not stored in the holder, and enables operation of the irradiation switch in a state where the photoacoustic probe is stored in the holder.

12. The object information acquiring apparatus according to claim 10, wherein the photoacoustic probe includes a contact detection sensor that detects whether the probe is in contact with the object.

13. The object information acquiring apparatus according to claim 12, further comprising a moving member that is disposed in the holder at a position that faces the contact detection sensor when the photoacoustic probe is stored in the holder,
wherein the controller moves the moving member to a position where the contact detection sensor can detect the state as a contact state when the irradiation switch is pressed.

14. The object information acquiring apparatus according to claim 10, wherein
the holder includes a storing detection sensor that detects whether the photoacoustic probe is stored in the holder, and
the controller sets conditions to irradiate the light from the irradiating end when the storing detection sensor detects that the photoacoustic probe is stored.

15. The object information acquiring apparatus according to claim 1, further comprising a display device configured to display information on the object interior.
